(19) United States
(12) Patent Application Publication
(10) Pub. No.: US 2016/0007867 A1
(43) Pub. Date: Jan. 14, 2016

(54) Combination Grounding Cuff

(71) Applicant: Marcus H. CRAWFORD, (US)

(72) Inventor: Marcus H. CRAWFORD, Kennesaw, GA (US)

(21) Appl. No.: 14/425,925

(22) PCT Filed: Jan. 15, 2014
(86) PCT No.: PCT/US14/11558

§ 371 Date: Mar. 4, 2015

(51) Int. Cl. A61B 5/022; A61B 18/16

(52) U.S. Cl. CPC A61B 5/02233; A61B 18/16

(57) ABSTRACT

The invention features a combined sphygmomanometer and a return electrode strip affixed within the sphygmomanometer's cuff. The return electrode strips sen es as a dedicated return ground for an electrosurgical device. The invention also contemplates use of one or more return electrodes positioned on the inner cuff of the sphygmomanometer and features their respective connection(s) with one or more respective electrosurgical device(s).

### FIELD OF THE INVENTION

The present invention relates, in general, to a combination grounding cuff featuring a sphygmomanometer combined with a return electrode strip dedicated for use as a return ground for an electrosurgical unit or device.

### BACKGROUND OF THE INVENTION

When patients are taken into the operating room to undergo surgery, a blood pressure cuff is normally placed in the patients preoperative area to enable monitoring of their blood pressure during surgery by an anesthesiologist. Once they are in the operating room, an electrosurgical unit having a handheld unit or device is normally used by the surgeon to perform surgery. The electrosurgical unit may also be used to stop any bleeding that occurs during the surgery.

During surgery, grounding pads are used to create a grounding path between the patient and ground. These pads come in different sizes, are normally disposable and are removed at the conclusion of the surgery. Some grounding pads are placed onto the patient with the help of an adhesive to keep them in place. When two surgeons are performing surgery on a patient, two separate grounding pads must be used with each one dedicated to each respective surgeon's electrosurgical device. These pads tend not be reusable.

### SUMMARY OF THE INVENTION

The present invention provides a combination grounding cuff featuring a sphygmomanometer and dedicated return electrode strip. The combination grounding cuff provides the dual function of providing a health care practitioner with blood pressure readings/monitoring while also providing a grounding path or return ground for an electrosurgical unit or device being used during a patient's surgery. The combination grounding cuff would work with patients regardless of size and would be reusable.

An aspect of an embodiment of the invention provides a combination grounding cuff having a sphygmomanometer which has an inner cuff surface for maintaining contact on a patient for taking blood pressure readings prior to, during and after surgery. The combination grounding cuff may also have one or more return electrode strip(s) which may be dedicated for use as a return ground for one or more electrosurgical unit(s)/device(s).

The one or more return electrode strip(s) may operate as a return ground for the one or more electrosurgical device(s) by collecting the current delivered to the patient by the one or more electrosurgical device(s) and carrying the current back to the one or more electrosurgical device(s).

The one or more return electrode strip(s) may be positioned and affixed on the sphygmomanometer's inner cuff surface, thereby enabling contact of the one or more return electrode strip(s) with the patient. The combination grounding cuff may further include one or more female return electrode adapter(s) which may be electrically connected to the one or more return electrode strip(s).

In another aspect of an embodiment of the present invention, the combination grounding cuff may additionally include one or more male return electrode adapters which may be connectively adapted for connection with the one or more female return electrode adapter(s). These one or more male return electrode adapters may be used to connect the combination grounding cuff to one or more electrosurgical unit(s). In one aspect of an embodiment of the present invention, a combination grounding cuff may have multiple return electrode strips affixed to its inner cuff surface in conjunction with multiple male & female return electrode adapter(s). This would enable the use of multiple electrosurgical units during a patient's surgery as each electrosurgical unit would be connected to a respective return electrode strip via respective male and female return electrode adapter(s). This is essential as each electrosurgical unit must have its own dedicated grounding pad or electrode in order to function properly.

In another aspect of an embodiment of the present invention, the combination grounding cuff may additionally include one or more electrode cord(s) and one or more corresponding female adapter(s) at an alternate end of the one or more electrode cord(s). The one or more female adapter(s) may be configured to connect with one or more electrosurgical unit(s).

In another aspect of an embodiment of the present invention, the one or more return electrode strip(s) of the combination grounding cuff may be demountably affixed to the inner cuff surface of the sphymomanometer. This would enable the removal and/or replacement of the return electrode strip(s).

In another aspect of an embodiment of the present invention, the combination grounding cuff may include one or more mounts affixed on the inner cuff surface of the sphygmomanometer. The one or more mounts may be configured to enable mounting of the one or more return electrode strip(s). In another aspect, the one or mounts may be configured to enable electrical connection between the one or more return electrode strip(s) and the one or more female return electrode adapter(s).

The one or more return electrode strip(s), in another aspect of an embodiment of the present invention, may be made of metalized plastic. Another aspect of an embodiment of the present invention may feature a flexible return electrode stop(s) thus enabling contouring of the return electrode strip(s) to make proper contact with the patient, as necessary.

In yet another aspect of an embodiment of the present invention, the one or more return electrode strip(s) may be capable of conducting electricity through the patient.

In yet another aspect of an embodiment of the present invention, the one or more return electrode strip(s) may be configured to enable its disconnection from the one or more female return electrode adapter(s).

In a further aspect of an embodiment of the present invention, the combination grounding cuff may include a sphygmomanometer with an inner cuff surface for maintaining contact on a patient and one or more return electrode strip(s), where the one or more return electrode strip(s) is dedicated for use as a return ground for at least one electrosurgical device.

The return electrode strip(s) may be positioned and affixed on the inner cuff surface of the sphygmomanometer, thereby enabling contact of the one or more return electrode strip(s) with the patient. The one or more return electrode strip(s) may be connected to an electrode cord which is connectively adapted to connect with one or more electrosurgical unit(s).

In a further aspect of an embodiment of the present invention, the one or more return electrode strip(s) may be demountably affixed to the inner cuff surface of the sphygmomanometer and, in another aspect, may be made of metalized plastic.

In a further aspect of an embodiment of the present invention, the one or more return electrode strip(s) may be capable of conducting electricity through the patient.

The combination grounding cuff as contemplated by the present invention provides the advantage of being reusable after cleaning and resulting in sayings to the practitioners or facilities using the device.

The combination grounding cuff also provides the advantage of enabling its use on multiple patients where the combination grounding cuff is cleaned between surgical settings.

The combination grounding cuff also has the advantage of providing a smaller and portable return electrode thereby eliminating the need for bigger electrodes which require contact with a sizable area of the patient's body.

Because the combination grounding cuff, may, in one aspect of an embodiment of the present invention, have a surface area that is similar to that of an adhesive patient return electrode, it could be used for any type of patient, including adults and children.

Another advantage of the combination grounding cuff as contemplated in the present invention is that one aspect of an embodiment of the present invention allows for the use of multiple electrosurgical devices.

A yet additional advantage of the combination grounding cuff, as contemplated by the present invention, is that a practitioner, while performing surgery on the patient may also be able to observe the patient's blood pressure readings while he/she is performing surgery with the electrosurgical device and, at the same time, have a return electrode provide the necessary grounding for the electrosurgical device.

Additional aspects, objectives, features and advantages of the present invention will become apparent from the following description of the preferred embodiments with reference to the attached drawings.

### BRIEF DESCRIPTION OF THE DRAWINGS

FIG. 1 illustrates a combination grounding cuff according to an aspect of an embodiment of the present invention.

FIG. 2 illustrates a combination grounding cuff according to another aspect of an embodiment of the present invention.

### DETAILED DESCRIPTION OF THE INVENTION

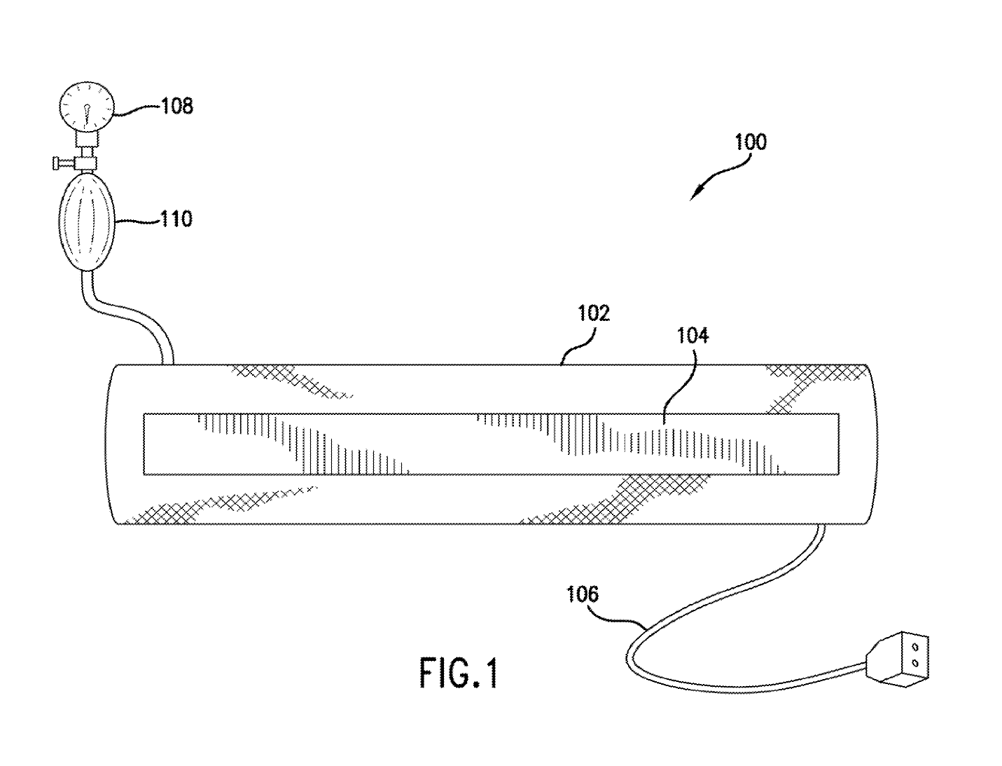

Referring now to FIG. 1 a combination grounding cuff 100, according to an aspect of an embodiment of the present invention, is shown. Here, combination grounding cuff 100 is shown with cuff 102 which may be used to wrap around a patient's arm, patient's preoperative area or other appropriate body part to obtain the patient's blood pressure readings. Cuff 102 may be inflated by use of bulb 110 which allows air to flow in and inflate cuff 102. Alternatively, cuff 102 may be inflated using known automated means or systems. The patient's blood pressure reading may then be read off dial 108. It should be noted that dial 108 may be aneroid or digital. Cuff 102 may be used to obtain the patient's blood pressure readings prior to, during and after surgery.

Combination grounding cuff 100 also includes return electrode strip 104 dedicated for use as a return ground for one or more electrosurgical device(s) (not shown) and may be affixed onto cuff 102. The strip is compatible with all electrosurgical generators (ESUs) used in surgical procedures.

In one aspect of an embodiment of the present invention, return electrode strip 104 may be removable or demountably affixed. In one aspect, a mount (not shown) secured onto the surface of cuff 102 may be used to affix return electrode strip 104. This enables return electrode strip 104 to be removed and/or replaced when necessary. In another aspect of an embodiment of the present invention, return electrode 104 may be made of metalized plastic. In yet another aspect it may be made of flexible electric current-conducting material to allow for contoured contact with the patient. Return electrode strip 104 may be electrically connected to electrode cord 106 which may be adapted to connect with an electrosurgical unit (not shown). In a yet further aspect of an embodiment of the present invention, the mount may be configured to enable electrical connection between return electrode strip 104 and electrode cord 106. Return electrode strip 104 acts as a return ground or return ground point for the electrosurgical unit (not shown). In doing so, return electrode strip 104 collects the current delivered to the patient during the surgery and transports the current away and back to the current generator (i.e. the electrosurgical unit) by way of electrode cord 106. The surgeon uses an active electrode to make contact with the tissue. The electric current flows from the active electrode, through the body to the return electrode, and then back to the electrosurgical generator.

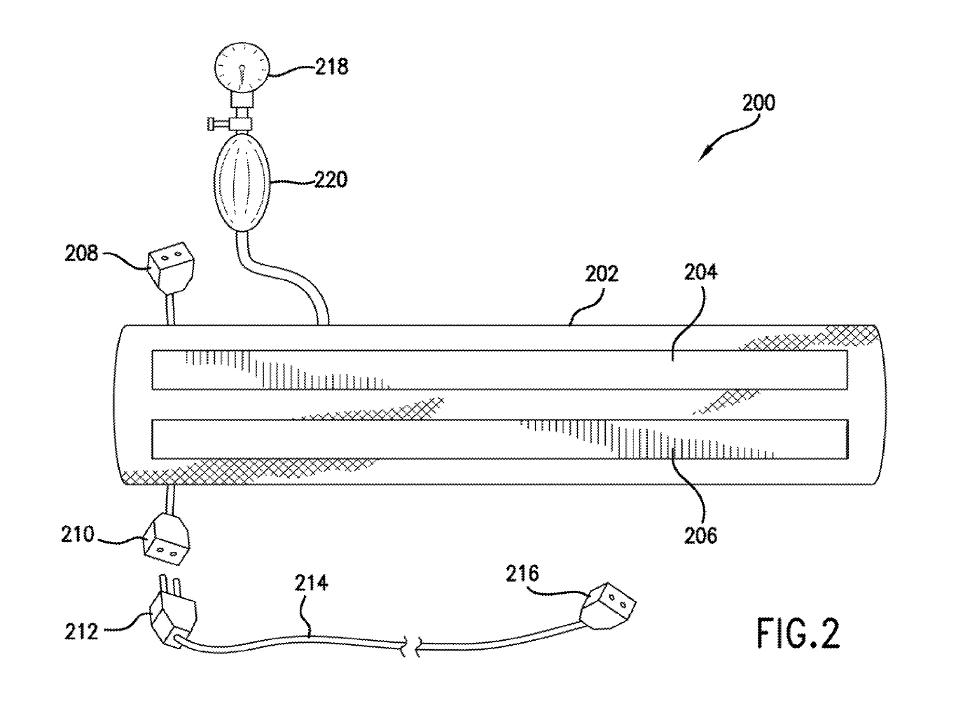

Referring now to FIG. 2, a combination grounding cuff 200, according to another aspect of an embodiment of the present invention, is shown. Here, device 200 is shown with cuff 202 used to take a patient's blood pressure reading with the help of bulb 218 and dial 220. Cuff 202 is shown with two return electrodes 204 and 206 affixed onto its inner surface as shown. Two return electrodes 204 and 206 enable the use of the two electrosurgical devices that may be used by two surgeons simultaneously performing surgery on a patient. It should be noted that the number of return electrodes is not limited to two as the device contemplated by the present invention, may incorporate additional return electrodes and thus enable the use of a similar number of electrosurgical devices during surgery.

Return electrodes 204 and 206 are both respectively connected with female return electrode adapters 208 and 210 which enable connection to an electrosurgical unit (not shown). Such a connection may be enabled, in one aspect, for return electrode to strip 204 by way of male return electrode adapter 212 having an electrode cord 214 and a female cord adapter 216 at the alternate end of cord 214. Female cord adapter 216 may be used to connect combination return device 200 with an electrosurgical unit (not shown).

Return electrodes 204 and 206 may, in another aspect of an embodiment of the present invention, be affixed onto cuff 202 by way of respective mounts (not shown). These mounts may be configured to enable electrical connection between return electrodes 204 and 206 with female return electrode adapters 208 and 210 respectively.

It should be noted that where multiple return electrode strips are used, a corresponding number of male & female return electrode adapters and electrode cords, having female adapters at each alternate end, may be used. It should also be noted that the combination grounding cuff contemplates a reversal of the male-female adapter pair configurations, as disclosed above, to female-male adapter pair configurations whereby for example, where a female adapter is disclosed, it may be replaced with a male adapter and where a male adapter is disclosed it may be replaced with a female adapter and so on.

Although this present invention has been disclosed with reference to specific forms and embodiments, it will be evident that a great number of variations may be made without departing from the spirit and scope of the present invention. For example, equivalent elements may be substituted for those specifically disclosed and certain features of the present invention may be used independently of other features—all without departing from the present invention as defined in the appended claims

What is claimed is:

1. A combination grounding cuff, comprising:
a sphygmomanometer, having an inner cuff surface for maintaining contact on a patient and taking blood pressure readings;
at least one return electrode strip, dedicated for use as a return ground for at least one electrosurgical device, said at least one return electrode strip being positioned and affixed on said inner cuff surface, thereby enabling contact of said at least one return electrode strip with said patient;
and
at least one female return electrode adapter, electrically connected to said at least one return electrode strip,
wherein the one return electrode strip collects current delivered to the patient and transports the current away from the patient.

2. The combination grounding cuff according to claim 1, further comprising at least one male return electrode adapter, connectively adapted to said at least one female return electrode adapter, said at least one male return electrode adapter being used to connect said grounding cuff to said at least one electrosurgical unit.

3. The combination grounding cuff according to claim 3, further comprising at least one electrode cord and at least one female adapter at an alternate end of said at least one electrode cord, said at least one female adapter being configured to connect with said at least one electrosurgical unit.

4. The combination grounding cuff according to claim 1 wherein said at least one return electrode strip is demountably affixed to said inner cuff surface.

5. The combination grounding cuff according to claim 1, further comprising a mount affixed on said inner cuff surface, said mount being configured to enable mounting of said at least one return electrode strip.

6. The combination grounding cuff according to claim 6, wherein said mount is configured to enable electrical connection between said at least one return electrode strip and said at least one female return electrode adapter.

7. The combination grounding cuff according to claim 1, wherein said at least one return electrode strip is made of metalized plastic.

8. The combination grounding cuff according to claim 1, wherein said at least one return electrode strip is capable of conducting electricity through said patient.

9. The combination grounding cuff according to claim 1, wherein said at least one return electrode strip is configured to be disconnected from said at least one female return electrode adapter.

10. An combination grounding cuff, comprising:
a sphygmomanometer having an inner cuff surface for maintaining contact on a patient and taking blood pressure readings; and
at least one return electrode strip, dedicated for use as a return ground for at least one electrosurgical device, said at least one return electrode strip being positioned and affixed on said inner cuff surface, thereby enabling contact of said at least one return electrode strip with said patient, wherein said at least one return electrode strip is connected to an electrode cord which is connectively adapted to connect with said at least one electrosurgical unit.

11. The combination grounding cuff according to claim 10, wherein said at least one return electrode strip operates as a return ground for said at least one electrosurgical device by collecting current delivered to said patient by said at least one electrosurgical device and carrying said current back to said at least one electrosurgical device.

12. The combination grounding cuff according to claim 10, wherein said at least one return electrode strip is demountably affixed to said inner cuff surface.

13. The combination grounding cuff according to claim 10, further comprising a mount affixed on said inner cuff surface, said mount being configured to enable mounting of said at least one return electrode strip.

14. The combination grounding cuff according to claim 13, wherein said mount is configured to enable electrical connection between said at least one return electrode strip and said electrode cord.

15. The combination grounding cuff according to claim 10, wherein said at least one return electrode strip is made of metalized plastic.

16. The combination grounding cuff according to claim 10, wherein said at least one return electrode strip is capable of conducting electricity through said patient.
